SEQUENCE LISTING (2 sequences)

SEQ ID NO: 1 - protein, 687 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 2 - DNA, 2651 bp, Homo sapiens
gcggacactcctctcggctcctccccggcagcggcggcggctcggagcgggctccggggctcgggtgcagcggccagcgggcctggcggcgaggattacccggggaagtggttgtctcctggctggagccgcgagacgggcgctcagggcgcggggccggcggcggcgaacgagaggacggactctggcggccgggtcgttggccgggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagaggtgagcactgcaacaaaaaggctgttttctctcggatctccaaatttaaaagcacaaggaatgattgtaccacacaaagtaatgtaaaacattaaaggactcattaaaaagtaacagttgtctcatatcatcttgatttattgtcactgttgctaactttcaggctcggaggagatgctcctcccaaaatgagttcggagatgatagcagtaataatgagacccccgggctccagctctgggccccccattcaggccgagggggctgctccggggggccgacttggtgcacgtttggatttggaggatccctgcactgccttctctgtgtttgttgctcttgctgttttctcctgcctgataaacaacaacttgggatgatcctttccattttgatgccaacctctttttatttttaagcggcgccctatagt

(19) United States
(12) Patent Application Publication
(10) Pub. No.: US 2010/0323380 A1
(43) Pub. Date: Dec. 23, 2010

(54) METHODS FOR DIAGNOSING BLOOD VESSEL REOCCLUSION

(75) Inventors: Navin K. Kapur, Hanover, MA (US); Richard H. Karas, Franklin, MA (US); Michael Mendelsohn, Boston, MA (US)

Correspondence Address: CLARK & ELBING LLP, 101 FEDERAL STREET, BOSTON, MA, 02110, US

(73) Assignee: Tufts Medical Center, Inc., Boston, MA (US)

(21) Appl. No.: 12/797,144

(22) Filed: Jun. 9, 2010

Related U.S. Application Data
(60) Provisional application No. 61/185,743, filed on Jun. 10, 2009.
(60) Provisional application No. 61/261,535, filed on Nov. 16, 2009.

(51) Int. Cl. C12Q 1/48

(52) U.S. Cl. 435/15

(57) ABSTRACT

The present invention features a method of diagnosing blood vessel reocclusion in a subject by detecting increased levels of sFLT-1 in a biological sample from the subject.

### CROSS REFERENCE TO RELATED APPLICATIONS

This application claims the benefit of U.S. Provisional Patent Application Nos. 61/185,743, filed Jun. 10, 2009 and 61/261,535, filed Nov. 16, 2009, each of which is hereby incorporated by reference.

### BACKGROUND OF THE INVENTION

In general, the invention relates to the use of soluble fins-like tyrosine kinase-1 (sFLT-1) for the diagnosis of reocclusion and restenosis.

Vascular reocclusion is a major long-term complication following surgical intervention of blocked arteries by percutaneous coronary intervention (PCI), atherectomy, laser angioplasty, and arterial bypass graft surgery. Functional tests, including invasive coronary angiography, myocardial perfusion imaging, and stress echocardiography, are often relied upon to detect reocclusion. Such studies incur significant healthcare costs and risk to patients. As a result, diagnostic methods to identify reocclusion in a patient and predict the risk of reocclusion development would be a valuable tool for improved stratification of patients.

Thus, there exists a need in the art for improved methods of diagnosing reocclusion.

### SUMMARY OF THE INVENTION

The present invention is directed to a method for diagnosing reocclusion of a blood vessel after reperfusion by measuring sFLT-1 levels in a subject.

In one aspect, the invention features a method of diagnosing reocclusion of a blood vessel after reperfusion of the blood vessel in a subject. The method includes obtaining a biological sample (e.g., a first biological sample) from a subject after reperfusion of a blood vessel and measuring the level of sFLT-1 present in the sample, wherein an increase in the level of sFLT-1 in the subject compared to the level of sFLT-1 in a subject not suffering from blood vessel reocclusion indicates reocclusion of a blood vessel in a subject. In one embodiment, a subject may be diagnosed with reocclusion of a blood vessel if the sFLT-1 level is increased above the 99^{th} percentile of reference standards or control values obtained from healthy individuals.

In certain embodiments, the method further includes obtaining a second biological sample from a subject having signs or symptom suggestive of coronary re-occlusion and measuring the level of sFLT-1 present in the second biological sample, wherein an increase in the level of sFLT-1 in the second biological sample compared to the first biological sample indicates reocclusion of a blood vessel in a subject. The second biological sample may be obtained less than 15 minutes, 15 minutes, 30 minutes, 1, 2, 3, 4, 5, 6, 12, 24, 48, 72 hours, or more after the first biological sample.

In some embodiments of the invention, the blood vessel is a coronary blood vessel (e.g., an artery).

In the present invention, reocclusion may occur in a subject that has been diagnosed with a cardiovascular condition such as acute coronary syndrome, atherosclerosis, transient ischemic attack, systolic dysfunction, diastolic dysfunction, aneurysm, aortic dissection, myocardial ischemia, angina pectoris, stable angina, unstable angina, acute myocardial infarction, acute ST-segment elevation myocardial infarction (STEMI), acute non-STEMI, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, and peripheral vascular disease.

In certain embodiments, the method of the invention may further include measuring the level of one or more additional biomarkers present in a biological sample obtained from a subject. The one or more additional biomarkers include, without limitation, annexin V, β-enolase, cardiac troponin I, cardiac troponin T, creatine kinase-Mb, glycogen phosphorylase-BB, heart-type fatty acid binding protein, C-reactive protein, growth differentiation factor 15, phosphoglyceric acid mutase-MB, S-100ao, myoglobin, actin, myosin, and lactate dehydrogenase.

The biological samples obtained in the methods of the present invention may be blood, serum, or plasma, and may be obtained after surgery.

In certain embodiments, the level of sFLT-1 in a subject (e.g., a control subject or reference standard) not suffering from reocclusion of a blood vessel is between 5-20 pg/ml (e.g., 5, 6, 7, 8, 9, 10, 11, 12, 13, 14, 15, 16, 17, 18, 19, or 20 pg/ml). In subjects diagnosed with reocclusion of a blood vessel, the level of sFLT-1 may be between 100-400 pg/ml (e.g., 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 pg/ml). Alternatively, the level of sFLT-1 in a sample from a subject being tested may be compared to a reference standard or reference level.

By “biological sample” is meant a bodily fluid (e.g., urine, blood, serum, plasma, or cerebrospinal fluid), tissue (e.g., cardiac tissue), or cell (e.g., cardiomyocyte) in which a polypeptide or nucleic acid molecule of the invention (e.g., sFLT-1) is normally detectable.

By “blood vessel” is meant arteries, veins, and capillaries. By “coronary blood vessel” is meant a blood vessel that delivers blood to the heart or transports blood away from the heart. Exemplary coronary blood vessels include (without limitation) the aorta, the right and left coronary arteries, the pulmonary vein, the pulmonary artery, the circumflex artery, the left anterior descending artery, and the vena cava.

By “cardiovascular condition” is meant disorders of the heart and vasculature, including, for example, atherosclerosis, transient ischemic attack, systolic dysfunction, diastolic dysfunction, aneurysm, aortic dissection, myocardial ischemia, acute myocardial infarction (AMI), acute ST-segment elevation myocardial infarction (STEMI), acute non-ST-segment elevation myocardial infarction (NSTEMI), angina pectoris, unstable angina (UA), and stable angina (SA), myocardial infarction, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, peripheral vascular disease, and peripheral artery disease.

By “biomarker related to a cardiovascular condition” is meant a biomarker that is known in the art to be derived from cardiac tissue and that is elevated in the circulation of subjects suffering from a cardiovascular condition. Exemplary biomarkers of a cardiovascular condition include, without limitation, annexin V, β-enolase, cardiac troponin I, cardiac troponin T, creatine kinase-MB, glycogen phosphorylase-BB, heart-type fatty acid binding protein, C-reactive protein, growth differentiation factor 15, phosphoglyceric acid mutase-MB, S-100ao, myoglobin, actin, myosin, and lactate dehydrogenase, or markers related thereto. See, e.g., Scirica, J. Am. Coll. Cardiol. 55:1403-1415, 2010.

By “fragment” is meant a portion of a polypeptide or nucleic acid molecule that contains at least 5%, 10%, 20%, 30%, 40%, 50%, 60%, 70%, 80%, 90%, 95%, or more of the entire length of a nucleic acid molecule or polypeptide (e.g., sFLT-1). A fragment may contain 10, 20, 30, 40, 50, 60, 70, 80, 90, 100, 200, 300, 400, 500, 600, or more nucleotides, up to 2651 nucleotides for sFLT-1 (SEQ ID NO: 2 and FIG. 2; GenBank Accession No. U01134.1). A fragment may contain 10, 20, 30, 40, 50, 60, 70, 80, 90, 100, 110, 120, 130, 140, 150, 160, 170, 180, 190, 200, or more amino acid residues, up to 687 amino acid residues for sFLT-1 (SEQ ID NO: 1 and FIG. 1; GenBank Accession No. AAC50060.1).

By “reference sample” is meant any sample, standard, standard curve, or level that is used for comparison purposes. A “normal reference sample” can be, for example, a prior sample taken from the same subject or a normal healthy subject; a sample taken from a subject that does not have reocclusion; a sample taken from a subject that is diagnosed with a propensity to develop reocclusion, but that does not yet show symptoms of the condition; a sample taken from a subject that has been treated for reocclusion; or a sample of a purified reference polypeptide or nucleic acid molecule of the invention (e.g., sFLT-1) at a known normal concentration. By “reference standard or level” is meant a value or number derived from a reference sample. A normal reference standard or level can be a value or number derived from a normal subject who does not have reocclusion. In certain embodiments, the reference sample, standard, or level may be, but need not be, matched to the sample subject by at least one of the following criteria: age, weight, body mass index (BMI), disease stage, and overall health.

By “reocclusion” or “restenosis” is meant the reoccurrence of stenosis (i.e., narrowing) of a blood vessel, leading to restricted blood flow. For example, reocclusion may pertain to a blocked or narrowed artery that has been treated to clear the blockage or occlusion and that has subsequently become reoccluded. Reocclusion is defined as a reduction in the circumference of the lumen of the blood vessel by, e.g., 10, 15, 20, 25, 30, 35, 40, 45, 50, 55, 60, 65, 70, 75, 80, 85, 90, 95, or 100%. Alternatively, reocclusion may refer to stenosis that results in reduced organ perfusion. Reocclusion may occur in a subject with, e.g., a cardiovascular condition.

By “reperfusion” is meant the restoration of blood flow to an organ or tissue. Reperfusion may involve, for example, thrombolytic therapy (e.g., administration of a thrombolytic agent (e.g., streptokinase, urokinase, alteplase, reteplase, or tenecteplase)), percutaneous coronary intervention (PCI) (e.g., angioplasty and stenting), or bypass surgery.

By “soluble fms-like tyrosine kinase-1,” “soluble FLT-1 (sFLT-1),” or “soluble VEGF receptor-1 (sVEGFR-1)” is meant the soluble form of the FLT-1 receptor (SEQ ID NOs: 1 and 2; FIGS. 1 and 2). As used herein, sFLT-1 also includes any isoform, fragment, or degradation product of sFLT-1.

By “subject” is meant a human or non-human (e.g., bovine, equine, canine, ovine, or feline) animal. The methods described herein are applicable to both human and veterinary disease. Further, while a subject is preferably a living animal, the invention described herein may be used in post-mortem analysis. For example, the term “subject” encompasses living humans that are receiving or being evaluated for medical care, including persons with no defined illness who are being examined for signs of disease.

Other features and advantages of the invention will be apparent from the detailed description and from the claims.

### BRIEF DESCRIPTION OF THE DRAWINGS

FIG. 1 is the amino acid sequence of sFLT1-1 (SEQ ID NO: 1).

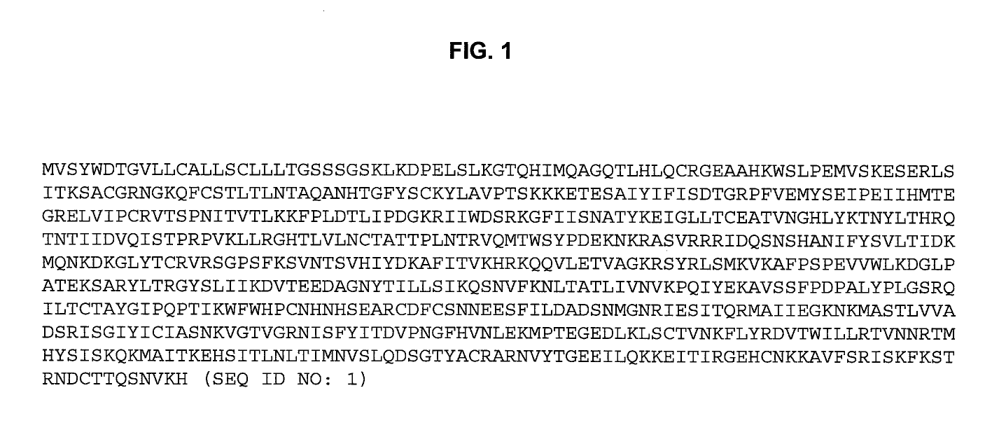

FIG. 2 is the nucleotide sequence of sFLT-1 (SEQ ID NO: 2).

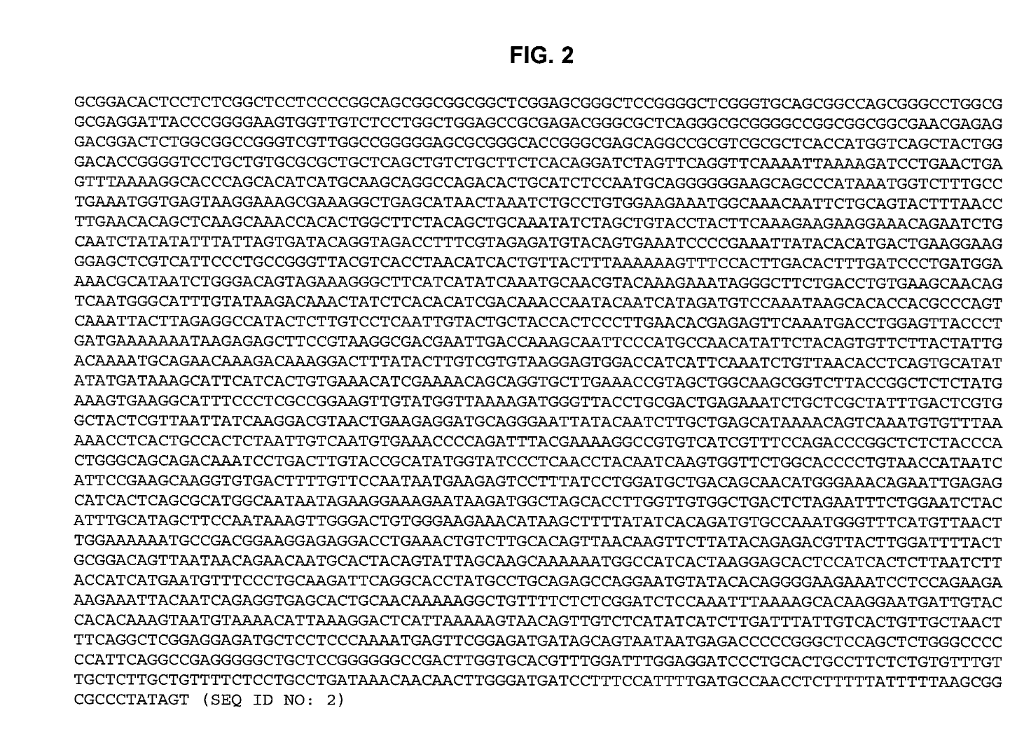

FIG. 3 is a bar graph showing sFLT-1 mRNA expression in human coronary endothelial cells exposed to hypoxia.

FIG. 4 is a graph that shows that hypoxia and thrombin induce rapid sFLT-1 release by human coronary endothelial cells in vitro.

FIG. 5 is a bar graph showing sFLT-1 mRNA expression in a mouse model of myocardial infarction.

FIG. 6 is a bar graph showing that left coronary ligation in a mouse model increases serum sFLT-1 levels within 30 minutes compared to sham controls.

FIG. 7 is a bar graph showing that sFLT-1 levels are significantly increased in subjects presenting with STEMI compared to control subjects.

FIG. 8 is a graph showing that serum sFLT-1 levels decline within 24 hours after percutaneous coronary revascularization and remain low at 48 hours follow-up, while CK-Mb and TnI levels were lower at presentation.

FIG. 9 is a bar graph showing that the mean sFLT-1 levels were significantly increased in subjects who presented within 60 minutes of symptom onset compared to patients who presented more than 360 minutes after symptom onset.

FIG. 10 is a bar graph showing that sFLT-1 levels exhibited 100% sensitivity for diagnosing acute STEMI across all time points after chest pain onset. In contrast, CK, CK-Mb, myoglobin, and TnI levels exhibited poor diagnostic sensitivity within 120 minutes of symptom onset.

FIG. 11 is a bar graph showing that sFLT-1 levels are elevated in patients with unstable angina (i.e., non-S′I′EMI).

FIG. 12 is a bar graph showing that, compared to pre-ablation values, serum sFLT-1 levels increased significantly within 15 minutes of septal artery ablation in patients with hypertrophic obstructive cardiomyopathy (HOCM). Levels gradually decreased by 30 and 60 minutes after ablation and returned to pre-ablation levels by 24 hours follow up. In contrast, CK-Mb and myoglobin levels were modestly increased within 15 minutes and continued to increase by 24 hours follow up.

FIG. 13 is a bar graph showing an increase in sFLT-1 in a subject that developed ST-elevations during a coronary stenting procedure. The graph shows that the sFLT-1 level doubled within minutes of arterial occlusion in the patient compared to the baseline level of sFLT-1.

FIG. 14 is a bar graph showing an increase in sFLT-1 levels 30 minutes after acute coronary occlusion using single whole-blood drop ELISA.

### DETAILED DESCRIPTION

We have discovered that serum levels of sFLT-1 are increased in patients suffering from blood vessel occlusion and that sFLT-1 is a biomarker for both occlusion and reocclusion of blood vessels. A major limitation of currently employed biomarkers of, for example, myocardial necrosis is their low sensitivity and lack of specificity for coronary occlusion. We have demonstrated that the sensitivity of serum sFLT-1 levels to detect coronary occlusion exceeds that of currently used biomarkers. Accordingly, the detection of sFLT-1 may be used as a diagnostic marker for cardiovascular conditions (e.g., blood vessel reocclusion) and for other acute vascular occlusive syndromes.

sFLT-1 and Reocclusion

As described herein, the present invention features methods for diagnosing or assessing the risk of blood vessel reocclusion by determining the level of sFLT-1 present in a subject after reperfusion. The reoccluded blood vessel may be a coronary blood vessel (e.g., the aorta, the right and left coronary arteries, the pulmonary vein, the pulmonary artery, the circumflex artery, the left anterior descending artery, or the vena cava) or any other vein or artery.

Blood vessel reocclusion may occur in subjects that have a cardiovascular condition. The cardiovascular condition may be, for example, acute coronary syndrome, atherosclerosis, transient ischemic attack, systolic dysfunction, diastolic dysfunction, aneurysm, aortic dissection, myocardial ischemia, angina pectoris, stable angina, unstable angina, acute myocardial infarction, acute ST-segment elevation myocardial infarction (STEMI), acute non-STEMI, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, or peripheral vascular disease.

### Diagnostic Methods

A subject experiencing reocclusion or a subject with a propensity to develop reocclusion may show an alteration (e.g., an increase of 5%, 10%, 20%, 30%, 40%, 50%, 60%, 70%, 80%, 90%, or more) in the expression of a sFLT-1 polypeptide. In one example, an increase in sFLT-1 expression in a sample taken from a subject compared to a normal reference sample is indicative of blood vessel reocclusion or a risk of developing the same. sFLT-1 can include full-length polypeptide, fragments, degradation products, alternatively spliced isoforms of the polypeptide, enzymatic cleavage products of the polypeptide, the polypeptide bound to a substrate or ligand, or free (e.g., unbound) forms of the polypeptide. Standard methods may be used to measure polypeptide levels in any bodily fluid including, but not limited to, urine, blood, serum, plasma, or saliva. Such methods include immunoassays, enzyme-linked immunoassays (ELISA), radioimmunoassays (RIAs), competitive binding assays, Western blotting using antibodies directed to sFLT-1 or fragments thereof (e.g., sc31173 antibody for sFLT-1 and sc-316 antibody for the cytoplasmic domain of FLT-1, both available from Santa Cruz Biotechnology, Inc.), and quantitative enzyme immunoassay techniques.

In certain embodiments, a subject experiencing reocclusion or a subject with a risk of developing reocclusion may show an increase in the expression of a nucleic acid (e.g., mRNA) encoding a sFLT-1 polypeptide. Methods for detecting such alterations are standard in the art and include, for example, Northern blotting and real-time PCR.

In another embodiment, hybridization techniques utilizing probes that are capable of detecting a sFLT-1 nucleic acid molecule, including genomic sequences or closely related molecules, may be used to detect a sFLT-1 nucleic acid sequence obtained from a subject experiencing reocclusion or a subject with a risk of developing reocclusion. The specificity of the probe and the stringency of the hybridization determine whether the probe hybridizes to naturally occurring sequences, allelic variants, or other related sequences. In one example, hybridization techniques may be used to monitor expression levels of a gene encoding a sFLT-1 polypeptide or fragments thereof.

The level of a sFLT-1 polypeptide, nucleic acid, or antibody can be measured once, and the level of may be compared to a control sample from a subject not suffering from blood vessel reocclusion. In other embodiments, the level of a sFLT-1 polypeptide, nucleic acid, or antibody can be measured at least two different times from the same subject and an alteration in the levels (e.g., an increase by 10%, 20%, 30%, 40%, 50%, 60%, 70%, 80%, 90%, or more) over time is used as an indicator of reocclusion or a risk of developing the same. For example, a second biological sample (e.g., a serum sample) may be obtained from a subject suffering from or at risk of suffering from reocclusion less than 15 minutes, 15 minutes, 30 minutes, 1, 2, 3, 4, 5, 6, 12, 24, 48, 72 hours, or more after the first sample.

In any of the diagnostic methods of the present invention, the level of, for example, sFLT-1 polypeptide, nucleic acid, or antibody may be measured to diagnosis or assess the risk of a subject developing reocclusion. For example, the level of sFLT-1 polypeptide present in a subject diagnosed with blood vessel reocclusion may be, for example, 100, 150, 200, 250, 300, 350, 400, 450, 500 pg/ml, or more, whereas the level of sFLT-1 in a subject not suffering from reocclusion of a blood vessel may be for example 50, 40, 30, 20, 25, 20, 15, 10, 5, 1 pg/ml, or less. Diagnostic methods can include measurement of absolute levels or relative levels of a sFLT-1 polypeptide, nucleic acid, or antibody as compared to a reference sample. In one example, an increase in the level of sFLT-1 polypeptide, nucleic acid, or antibody as compared to a normal reference, is considered a positive indicator of reocclusion or a propensity to develop the same.

In other embodiments of the present invention, the biological activity of sFLT-1 may be measured, where an increase in activity relative to a sample taken from a control subject is diagnostic of reocclusion. For example, binding assays to measure sFLT-1 binding kinetics to angiogenic peptides could be performed to quantitate sFLT-1 function. Other functional assays known to those skilled in the art could also be performed.

The diagnostic methods described herein can be used individually or in combination with any other diagnostic method for a more accurate diagnosis of the presence of, severity of, or predisposition to blood vessel reocclusion in a subject. Such diagnostic methods include, for example, electrocardiography, echocardiography, coronary angiography, chest radiography, physical examination, histopathological examination, blood chemistry analysis, computed tomography, cytological examination, magnetic resonance imaging, and identification of other diagnostic biomarkers (e.g., annexin V, β-enolase, cardiac troponin I, cardiac troponin T, creatine kinase-Mb, glycogen phosphorylase-BB, heart-type fatty acid binding protein, C-reactive protein, growth differentiation factor 15, phosphoglyceric acid mutase-Mb, S-100ao, myoglobin, actin, myosin, and lactate dehydrogenase).

### Diagnostic Kits

The invention also provides for a diagnostic test kit. For example, a diagnostic test kit can include polypeptides (e.g., antibodies that specifically bind to sFLT-1 or fragments thereof) and components for detecting and/or evaluating binding between the polypeptide (e.g., antibody) and sFLT-1. Alternatively, the kit can include a sFLT-1 polypeptide or sFLT-1 fragment for the detection of sFLT-1 antibodies present in the serum or blood of a subject sample. In another example, diagnostic kits of the invention may be used to identify an alteration in the level of a sFLT-1 polypeptide relative to a reference, such as the level present in a normal control (e.g., the level in a subject not experiencing blood vessel reocclusion). Such a kit may include a reference sample or standard curve indicative of a positive reference or a normal control reference.

For detection, either the antibody or the sFLT-1 polypeptide is labeled, and either the antibody or the sFLT-1 polypeptide is substrate-bound, such that the polypeptide-antibody interaction can be established by determining the amount of label attached to the substrate following binding between the antibody and the sFLT-1 polypeptide. Conventional immunoassays (e.g., ELISA) may be used for detecting antibody-substrate interactions and can be provided with the kit of the invention. The polypeptides of the invention can be detected in a biological sample, such as blood, plasma, or serum.

The diagnostic kit may include instructions for the use of the kit. In one example, the kit contains instructions for the use of the kit for the diagnosis of blood vessel reocclusion or a risk of developing the same. In yet another example, the kit contains instructions for the use of the kit to monitor therapeutic treatment, dosage regimens, or subjects recovering from vascular or cardiac surgery (e.g., angioplasty).

### Subject Monitoring

The diagnostic methods described herein can also be used to monitor the onset of reocclusion in a subject during therapy or to determine the dosage(s) of therapeutic compound(s) needed to treat the condition. In this embodiment, the levels of sFLT-1 polypeptide, nucleic acid, or antibody may be measured repeatedly as a method of diagnosing reocclusion and also monitoring the treatment, prevention, or management of reocclusion. To monitor the progression of blood vessel reocclusion in a subject, subject samples may be compared to reference samples taken early in the diagnosis of the disorder. In one example, levels of sFLT-1 polypeptide can be monitored in a subject that has been diagnosed with reocclusion. A decrease of sFLT-1 polypeptide in a subject being treated for reocclusion indicates an improvement in or the absence of reocclusion. Such monitoring may be useful, for example, in determining proper dosages for therapeutic treatment or in assessing the efficacy of a particular therapeutic regimen.

In addition, the diagnostic methods of the invention may be used to monitor a subject that has risk factors for reocclusion (e.g., a subject having a family history of a cardiovascular disease or subject that has undergone vascular or cardiac surgery (e.g., angioplasty)). In such an example, therapeutic methods (e.g., stenting, additional angioplasty, or brachytherapy) can then be used proactively to promote vascular health and/or to prevent reocclusion from developing or further progressing.

### EXAMPLES

The present invention is illustrated by the following examples, which are in no way intended to be limiting of the invention.

### Example 1

### sFLT-1 Expression in Human Coronary Endothelial Cells

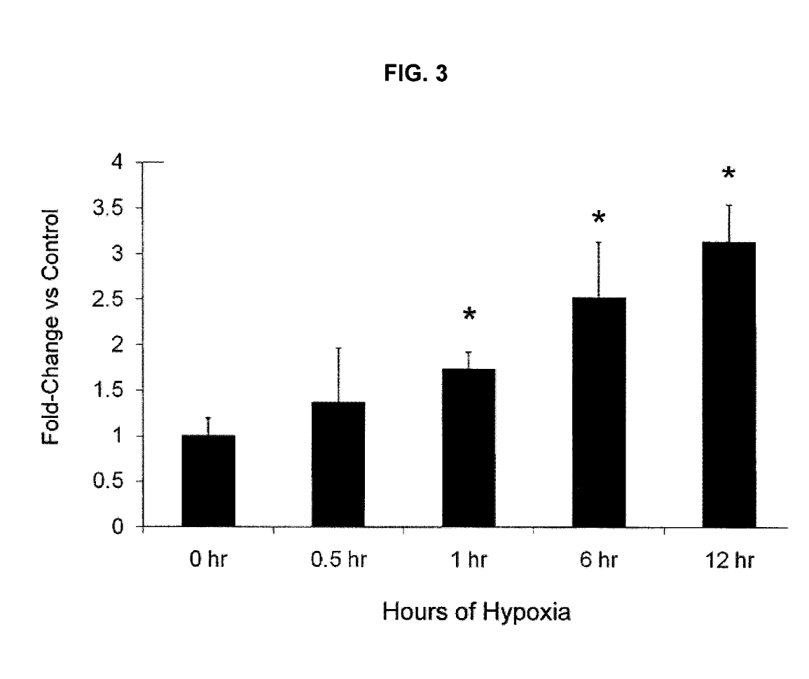

To explore the cellular origin of sFLT-1, human coronary endothelial (HCE) cells and smooth muscle cells (HSMC) were rendered hypoxic. Within 1 hour, sFLT-1 mRNA expression in HCE cells increased by 173%±35% (p=0.03) (FIG. 3). By 12 hours, sFLT-1 mRNA and protein expression in hypoxic HCE cells increased by 313%±15% (p<0.01) and 256%±17% (p<0.001), respectively. To simulate the effects of reperfusion on sFLT-1 expression, HCE cells were exposed to 12 hours of hypoxia, then 6 hours of normoxia. After normoxic rescue, sFLT-1 protein expression decreased by 36% (p<0.01 versus hypoxic HCE). Hypoxia failed to induce sFLT-1 mRNA or protein expression in HSMC.

### Example 2

### sFLT-1 Release Kinetics In Vitro

To simulate the microenvironment of an acute coronary occlusion, human coronary artery endothelial cells (HCAECs) were exposed to hypoxia and exogenous thrombin.

HCAECs (Cell Applications Inc.) were cultured to near confluence using normal humidified tissue culture incubators with 5% CO₂. For hypoxia experiments, gas-tight modular incubator chambers (Billups-Rothenberg, Del Mar, Calif., USA) were flushed with a gas mixture containing 5% CO₂ and 95% N₂ for 15 minutes. Serum-free media (Dulbeco's) was incubated in the chamber for 18 hours under hypoxic conditions and transferred to culture dishes containing nearly confluent cells. For hypoxia-thrombin co-stimulation studies, HCAECs were rendered hypoxic as described in the presence and absence of recombinant human thrombin (5 U/mL; Sigma-Aldrich). HCAECs and conditioned media were harvested at various time points for protein and mRNA expression analysis.

To monitor mRNA expression, RNA from cultured cells was extracted using TRIZOL Reagent (Invitrogen, Carlsbad, Calif.). Real-time quantitative polymerase chain reaction (PCR) was performed with a 7900HT Sequence Detection System (Applied Biosystems, Foster City, Calif.). Triplicate samples were subjected to reverse transcription and real-time PCR with TaqMan One-Step RT-PCR Master Mix Reagents with gene-specific primers and probes designed by Primer Express software (Applied Biosystems; Forward primer for sFLT-1: AGGTGAGCACTGCGGCA; Reverse primer: ATGAGTCCTTTAATGTTTGAC). sFLT-1 gene expression was normalized to total RNA content by quantification of GAPDH gene expression.

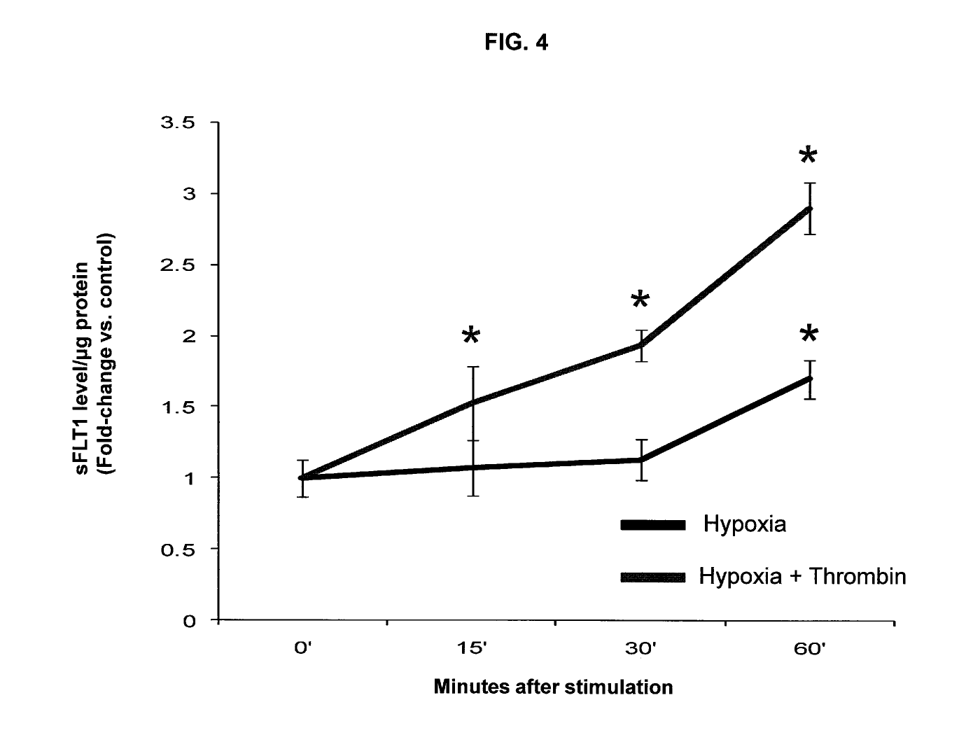

Co-stimulation with both hypoxia and thrombin induced a significant increase in sFLT-1 mRNA expression similar to hypoxia alone; however, sFLT-1 protein release into the conditioned media was significantly increased within 15 minutes of co-stimulation (FIG. 4). These findings confirm that hypoxia induces sFLT-1 expression in coronary endothelial cells and suggest that sFLT-1 protein release occurs within minutes in the presence of both hypoxia and thrombin.

### Example 3

### sFLT-1 Expression in a Mouse Model

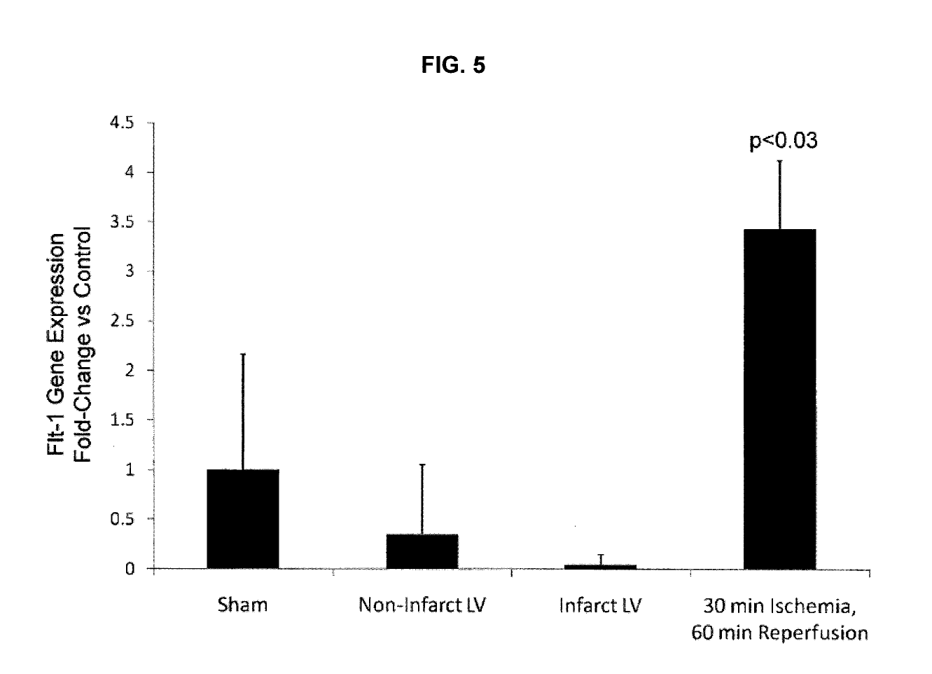

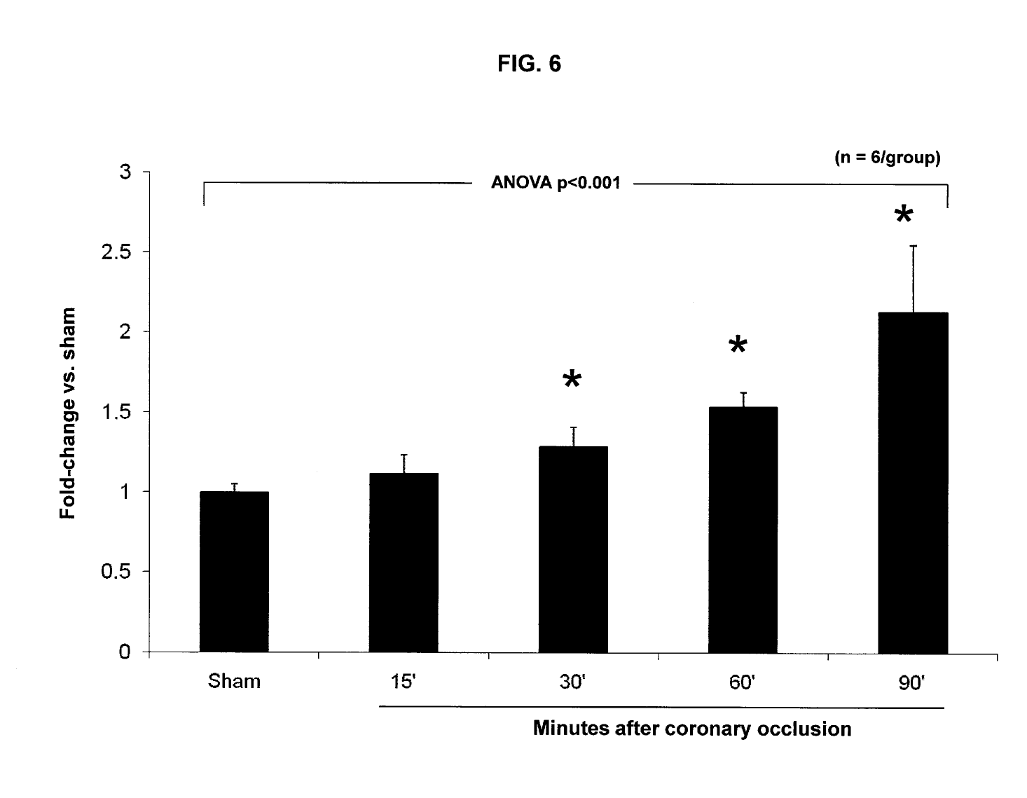

We studied sFLT-1 expression in a mouse model of myocardial infarction (MI). After 1 hour of left coronary ligation in 10-week old male wild-type mice (n=6/group), left ventricular sFLT-1 mRNA levels were increased (p<0.01 MI versus sham) (FIG. 5), while aortic and lung mRNA expression were unchanged (data not shown). Serum sFLT-1 levels were also elevated in MI (170±71 versus 541±107 pg/mL, sham versus MI, p<0.002) (FIG. 6).

### Example 4

### sFLT-1 Expression in Acute STEMI Patients

In the following experiments, we demonstrated that sFLT-1 is a biomarker of coronary artery occlusion during the acute phase of STEMI.

Patient Population

We enrolled 30 patients presenting to Tufts Medical Center with ST-segment elevation myocardial infarction (STEMI) within 12 hours of symptom onset who were referred for emergent percutaneous coronary angiography and intervention. All STEMI subjects were required to have active chest pain and documented ST-segment elevation on arrival to the catheterization laboratory. Other inclusion criteria were as follows: age >18 years and <90 years of age; sinus rhythm; and the ability to provide informed consent. Patients with hemodynamic or clinical instability; perceived interference with standard clinical care of patients; unsuccessful reperfusion; pregnancy; active or remote cancer; renal failure (estimated glomerular filtration rate <30); or liver transaminases greater than 2 times the upper limit of normal were excluded. All eligible patients who agreed to enroll had blood sampled at the time of arterial sheath insertion for diagnostic angiography and at 24 and 48 hours after revascularization.

Patients referred for clinically indicated septal alcohol ablation due to hypertrophic obstructive cardiomyopathy (HOCM) also were enrolled. All HOCM subjects were required to have a significant pressure gradient across the left ventricular outflow tract exceeding 50 mm Hg at rest or with provocation. The same exclusion criteria listed above were applied to HOCM subjects referred for septal ablation. Blood sampling was performed at the time of arterial sheath insertion for diagnostic angiography, and at 15, 30, and 60 minutes, and 24 and 48 hours after ablation. At each time point, total creatine kinase (CK), creatine kinase-Mb (CK-Mb), myoglobin, and troponin-I (TnI) levels were measured.

The baseline characteristics of the overall study population are provided in Table 1. Compared to 25 healthy control subjects, patients with STEMI had more cardiovascular risk factors (p<0.05). Groups did not differ in age, gender, or race.

TABLE 1
Baseline Characteristics of the Overall Study Population

| Variable | STEMI | Healthy Control | p-value |
|---|---|---|---|
| Age (years) | 61 ± 15 | 54 ± 8 | <0.01 |
| Male Gender, n (percent) | 21 (70%) | 11 (55%) | NS |
| Caucasian, n (percent) | 28 (93%) | 17 (85%) | NS |
| BMI, kg/m² | 30 ± 7 | 32 ± 8 | NS |
| Hypertension, n (percent) | 18 (60%) | 0 (0%) | <0.001 |
| Diabetes mellitus, n (percent) | 6 (20%) | 0 (0%) | <0.001 |
| Dyslipidemia, n (percent) | 17 (57%) | 0 (0%) | <0.001 |
| Active smoking, n (percent) | 14 (47%) | 0 (0%) | <0.001 |
| PVD, n (percent) | 3 (10%) | 0 (0%) | <0.001 |
| CAD, n (percent) | 7 (23%) | 0 (0%) | <0.001 |
| Prior MI, n (percent) | 3 (10%) | 0 (0%) | <0.001 |

Clinical characteristics of patients with STEMI are presented in Table 2. Among the 30 patients presenting with STEMI, intracoronary thrombus was evident by angiography in 55% of the patients, while thrombolysis in myocardial infarction (TIMI) 0 or 1 flow was observed in 93% of the patients. Unfractionated heparin and eptifibatide infusions were administered in 93% of cases with bivalirudin used as the primary anticoagulant in the remaining 7%.

TABLE 2
Clinical Characteristics of Patients with STEMI

| Variable | Mean ± SD |
|---|---|
| Time from Chest Pain Onset to | 262.8 ± 198 |
| Presentation (minutes) | |
| Reperfusion Time (minutes) | 61 ± 31 |
| Admission LVEF (Mean ± SD) | 44 ± 14% |
| Killip Classification (Mean ± SD) | 1.7 ± 0.09 |
| Thrombolysis in Myocardial Infarction | 0.43 ± 0.46 |
| (TIMI) Flow | |
| Serum Chemistries on Admission | |
| Sodium | 137 ± 3.5 |
| Creatinine | 0.9 ± 0.3 |
| Glucose | 131 ± 33 |
| White Blood Cell Count | 11.1 ± 2.9 |
| Hemoglobin | 13.3 ± 1.7 |
| Platelets | 244 ± 78 |
| Medications on Admission | |
| Aspirin | 30% |
| Clopidogrel | 7% |
| Beta-blockers | 37% |
| ACE-Inhibitor | 27% |
| Calcium Channel Blocker | 10% |
| Angiotensin Receptor Blocker | 7% |
| Aldosterone Antagonist | 3% |
| Diuretic | 17% |
| Anti-Dyslipidemic Agent | 37% |

Control subjects were healthy volunteers with no prior medical history and taking no medications. Subjects were required to be between 21 and 80 years of age. Serum samples were obtained as a one-time lab draw in a clinical research center.

All physicians were blinded to the results of the serum analysis. Patients received standard clinical care for STEMI during their index hospitalization, including serial electrocardiograms (ECGs), testing of cardiac biomarkers, and pharmacologic therapy including aspirin, clopidogrel, anticoagulation with unfractionated heparin or bivalirudin, glycoprotein IIb/IIIa receptor inhibitors, HMG-CoA reductase inhibitors, beta-blockade, and ACE-inhibition as clinically indicated. The institutional review board of Tufts Medical Center approved this study, and all patients provided written informed consent.

Biomarker Assays

Blood samples were collected using a serum separator tube (SST) and allowed to clot for 30 minutes prior to centrifugation at 2000×g for 15 minutes. Serum samples were immediately stored at −20° C. sFLT-1 and myoglobin levels were measured in duplicate for each serum sample using a commercially available quantitative sandwich enzyme immunoassay kits (ELISA; soluble sFLT-1, R&D Systems; myoglobin, Calbiotech, Inc.) according to the manufacturers' instructions. Total CK, CK-Mb, and ultra-sensitive TnI levels were measured by the central clinical laboratory using the Advia Centaur Immunoassay System (Siemens).

Statistical Analysis

Data are expressed as means±SD. Normality of distribution was assessed using Kolmogorov-Smirnof, Shapiro-Wilk tests, and Q-Q plots. Pair-wise comparisons were made using ANOVA for continuous variables, and the chi-squared and/or Fisher's exact tests for categorical values. Post hoc comparisons were made using the Schefe method where appropriate. ANOVA with repeated measures was used to examine change in outcome variables over time. When a significant main effect was detected, appropriate post hoc comparisons were made. Pearson's correlation coefficients were used to assess relationships between variables of interest. Stepwise multiple regression analysis was performed to examine correlates of sFLT-1 in our cohort with STEMI. Variables entered into the model included traditional cardiovascular risk factors (e.g., age, gender, presence/absence of hypertension, diabetes, hyperlipidemia, family history, and smoking status) and history of coronary artery disease. The fit of the regression models were checked by the Hosmer-Lemeshow test for goodness of fit. Area under the receiver operator characteristic (ROC) curve was also examined to determine the predictive power of sFLT-1 for detecting presence of STEMI. All statistical analyses were performed using SigmaStat Version 3.1 (Systat Software, Inc) and Statistical Package for the Social Sciences (SPSS, v 16.0.1, SPSS, Inc., Chicago, Ill.). p<0.05 was selected to denote significant differences.

Results

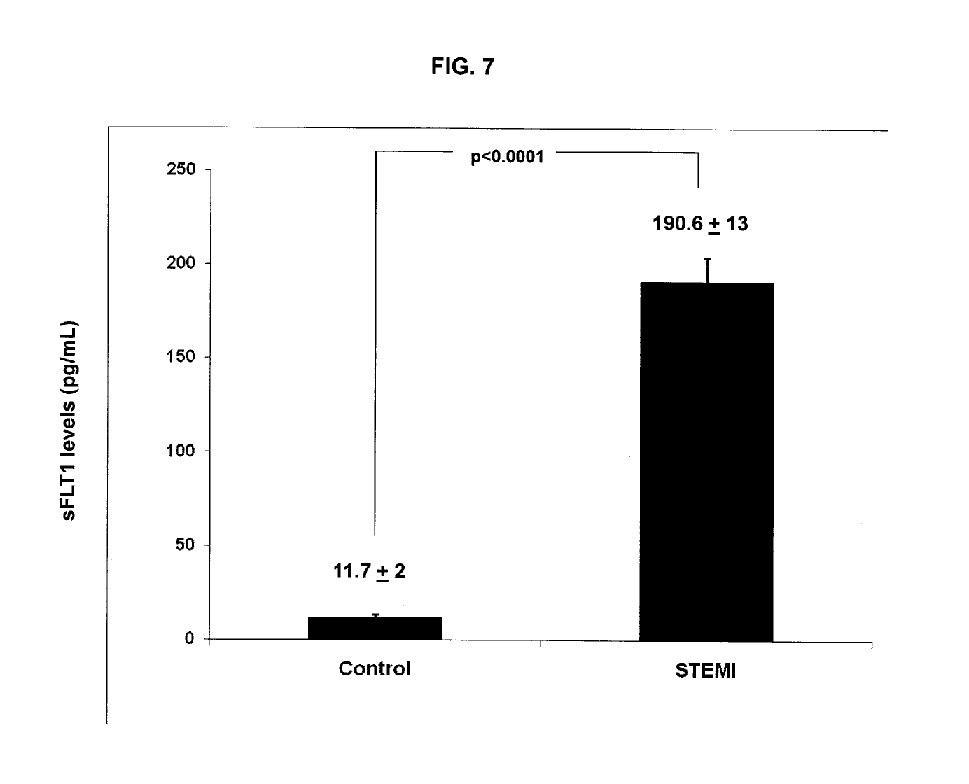

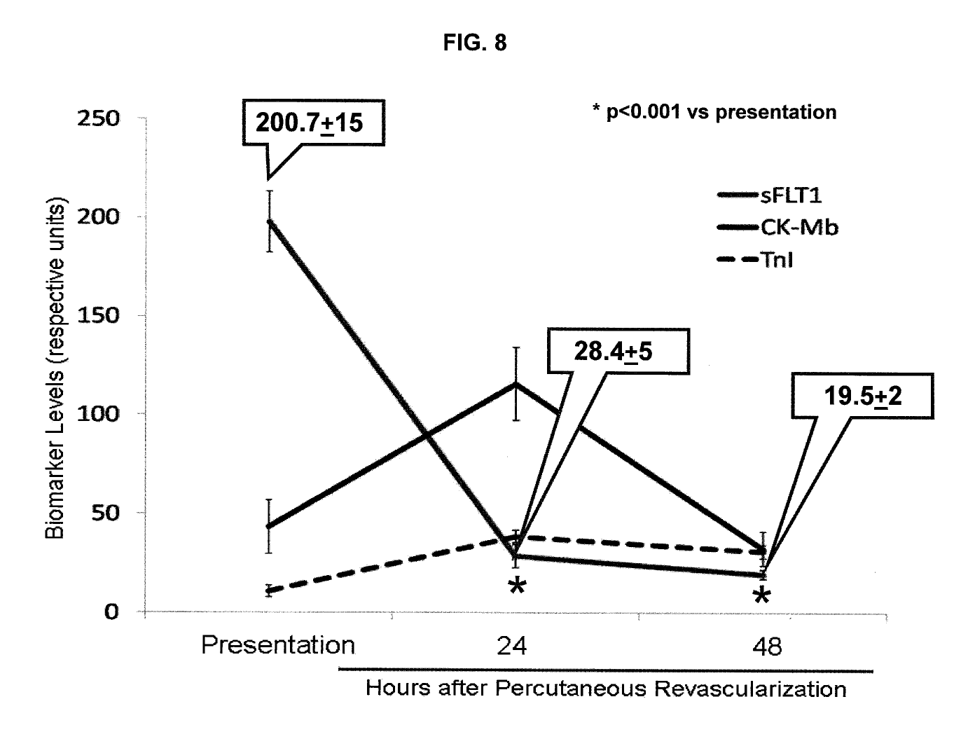

Compared to healthy controls, patients with STEMI exhibited markedly elevated sFLT-1 levels at the time of presentation (p<0.05) (FIG. 7). Group differences in sFLT-1 remained after co-varying for group differences in age and cardiovascular risk factors. According to RM ANOVA, sFLT-1 levels were highest at time of presentation and significantly decreased over time (p<0.05). Values at 24 and 48 hours of follow up were significantly lower than values at time of presentation (FIG. 8). sFLT-1 values at 48 hours did not differ from values at 24 hours of follow-up (p>0.05). Myoglobin levels significantly decreased over time (p<0.05) with significantly lower values at 24 and 48 hours of follow-up after percutaneous revascularization (data not shown). Myoglobin values at 48 hours did not differ from values at 24 hours of follow up (p>0.05). There was a significant increase in total CK from time of presentation (p<0.05) (data not shown). CK values at 24 hours of follow up were higher than values at time of presentation and 48 hours of follow-up (p<0.05) (data not shown). There was a significant increase in CK-Mb values over time (p<0.05) (FIG. 8). Values at 24 hours of follow up were significantly higher than values at time of presentation and 48 hours after presentation (p<0.05). Ultra-sensitive TnI values were significantly higher at 24 and 48 hours of follow-up compared to the values at presentation (p<0.05).

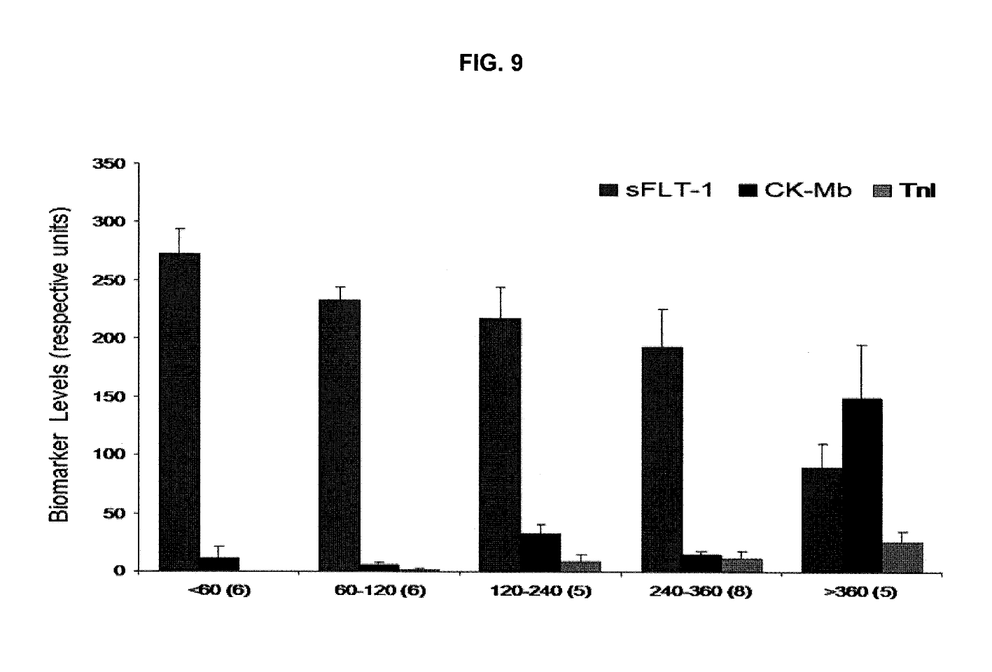

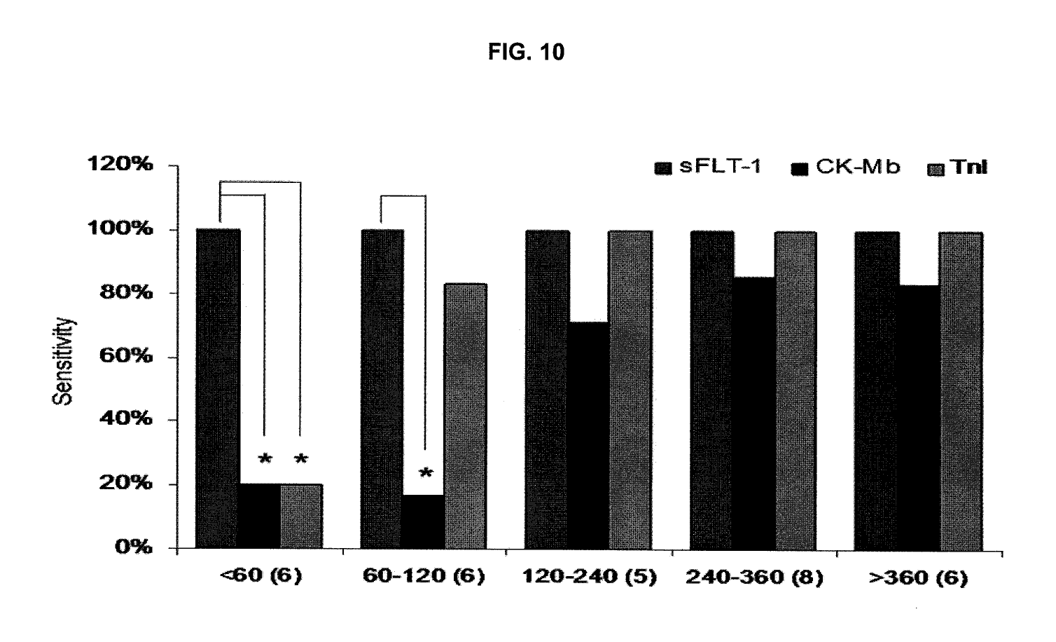

At presentation, 100% of STEMI patients had elevated sFLT-1 levels greater than the 99^{th} percentile of values measured in normal healthy controls (>15 pg/mL), whereas only 70% and 83% had levels of CK-Mb>5 ng/mL and ultra-sensitive TnI >0.05 ng/mL (previously established clinical cut-points), respectively. Using the aforementioned cut-point of 15 pg/mL optimized both specificity (99.5%) (FIG. 9; Table 3) and sensitivity (100%) (FIG. 10; Table 4). Area under the ROC curve for sFLT-1 discerning STEMI from healthy controls was 1.00 (p<0.001).

TABLE 3
sFLT-1 levels across all time points from symptom onset

| | Minutes from chest pain onset (n = number) | | | | |
|---|---|---|---|---|---|
| | <60 (6) | 60-120 (6) | 120-240 (5) | 240-360 (8) | >360 (5) |
| sFLT-1 (pg/ml) | 272.39 ± 21 | 233.4 ± 11 | 217.86 ± 26 | 193.26 ± 33 | 89.72 ± 20 |
| CK-Mb (ng/ml) | 11.74 ± 9 | 5.53 ± 2 | 32.96 ± 8 | 14.83 ± 3 | 149.03 ± 47 |
| Ultra-sensitive | 0.05 ± 0.02 | 1.82 ± 1.2 | 9.04 ± 6.0 | 11.91 ± 6.4 | 25.92 ± 8.7 |
| TnI (ng/ml) | | | | | |

TABLE 4
sFLT-1 levels exhibited 100% sensitivity for diagnosing acute STEMI

| | Minutes from chest pain onset (n = number) | | | | |
|---|---|---|---|---|---|
| | | | | 240-360 | |
| | <60 (6) | 60-120 (6) | 120-240 (5) | (8) | >360 (6) |
| sFLT-1 | 100.00% | 100.00% | 100.00% | 100.00% | 100.00% |
| CK-Mb | 20.00% | 50.00% | 80.00% | 85.71% | 83.3% |
| Ultra- | 20.00% | 83.33% | 100.00% | 100.00% | 100.00% |
| sensitive | | | | | |
| TnI | | | | | |
| (ng/ml) | | | | | |

As a first step toward understanding the effect of duration of coronary artery occlusion on sFLT-1 release, we used time from symptom onset as a clinical marker for onset of coronary artery occlusion. Mean sFLT-1 levels were significantly increased in subjects who presented within 60 minutes of symptom onset compared to patients who presented more than 360 minutes after symptom onset (p<0.05) (data not shown). Across groups of chest pain, there were no significant group differences in total CK (p=0.06), ultra-sensitive TnI (p=0.06), or myoglobin (p=0.61). There were significant group differences in CK-Mb (p<0.05). CK-Mb levels were higher at >360 minutes from chest pain onset compared to all other time points (p<0.05). Across all time points of minutes after chest pain onset, sFLT-1 levels exhibited 100% sensitivity for diagnosing an acute STEMI. In contrast, total CK, CK-Mb, myoglobin, and ultra-sensitive TnI levels exhibited poor diagnostic sensitivity within 120 minutes of symptom onset.

According to stepwise multiple regression analysis, cardiovascular risk factors were not significant correlates of sFLT-1 levels in patients with STEMI, as none of the selected variables (i.e., age, gender, age, gender, presence/absence of hypertension, diabetes, hyperlipidemia, family history, smoking status, and CAD) entered into the linear model (p>0.05). Moreover, sFLT-1 values at the time or presentation did not differ between STEMI patients with versus without aforementioned cardiovascular risk factors (p>0.05). There was no correlation between sFLT-1 at the time of presentation with indices of renal function or left ventricular ejection fraction on admission (p>0.05). Serum sFLT-1 levels correlated inversely with markers of myocardial necrosis, such as peak total CK and CK-Mb (Table 5).

TABLE 5
Levels of sFLT-1 demonstrated an inverse correlation with
markers of myocardial injury and predictors of mortality in STEMI
including peak total CK, peak CK-Mb, and time from chest pain onset

| Variable | Pearson's R-value | p-value |
|---|---|---|
| Peak CK | −0.51 | 0.004 |
| Peak CK-Mb | −0.38 | 0.04 |
| Peak Troponin-I | −0.26 | 0.16 |
| Peak Myoglobin | −0.21 | 0.2 |
| Door-to-Balloon Time | −0.44 | 0.02 |

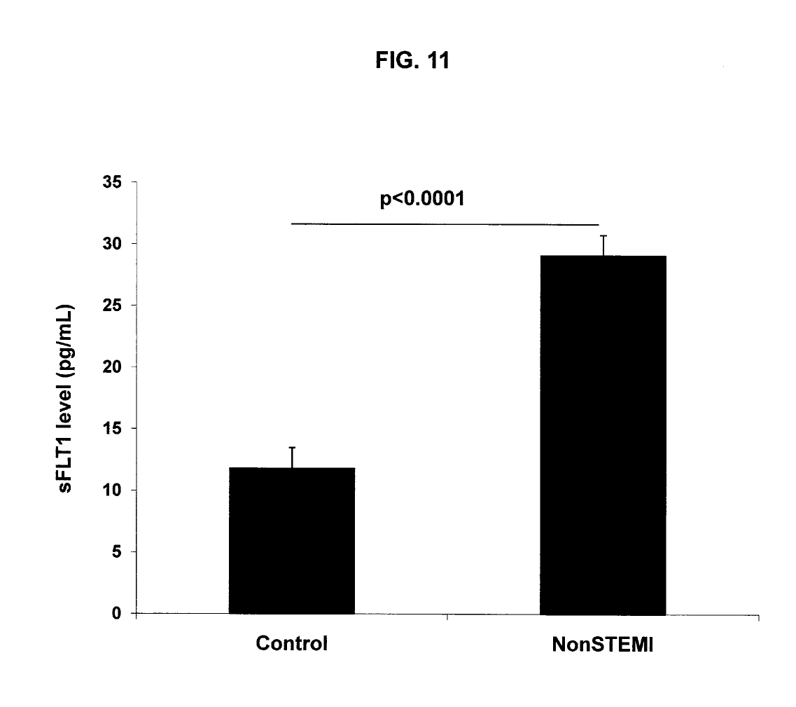

As shown in FIGS. 9 and 10, sFLT-1 levels were significantly associated with time from symptom onset, with higher levels observed in patients who present early and relatively lower levels in patients who present later. We also found that sFLT-1 levels were elevated in subjects with other cardiovascular conditions, such as unstable angina (i.e., non-STEMI) (FIG. 11).

To control for an interaction between sFLT-1 ELISA measurements and anticoagulant therapy during percutaneous coronary intervention, serum samples from normal human subjects were treated with 4 units/mL unfractionated heparin, for 90 minutes followed by sFLT-1 ELISA. No difference in sFLT-1 levels was observed between heparin-treated and non-heparin treated samples (9.6±0.9 versus 8.8±0.8 pg/mL; n=4/group, p=0.2) (data not shown).

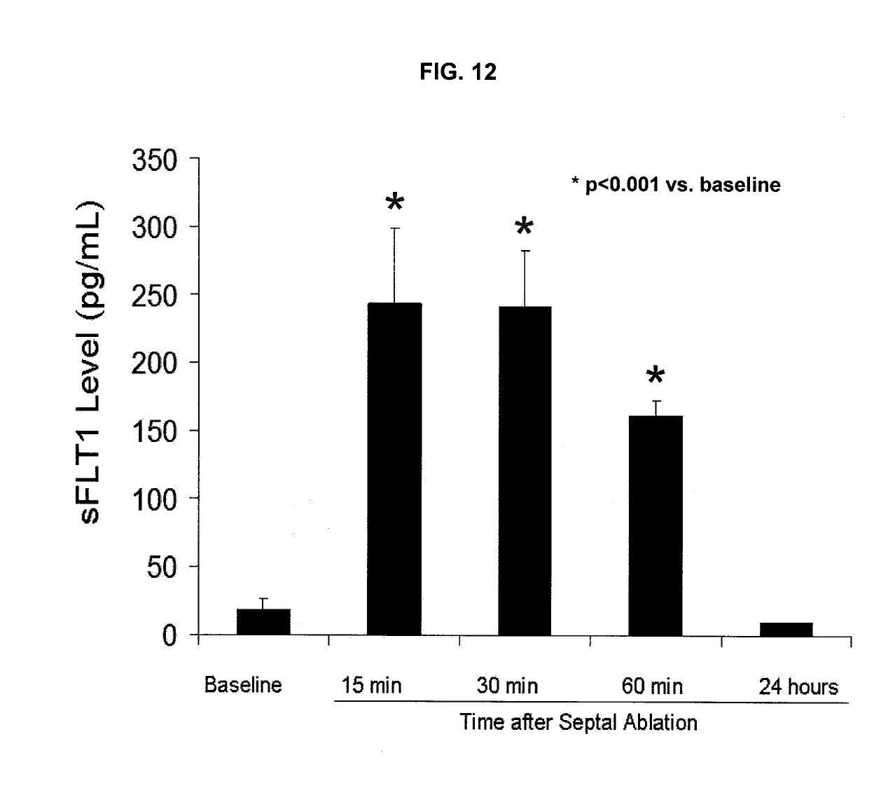

To further explore the kinetics of sFLT-1 release, we prospectively measured serum levels in three patients with hypertrophic obstructive cardiomyopathy (HOCM) referred for septal artery ablation. During this procedure, the septal artery is occluded with a balloon catheter, followed by ethanol injection into the myocardium supplied by the selected septal artery. In this manner, we were able to measure sFLT-1 levels before and during the acute phase of coronary artery occlusion. Compared to preocclusion levels, sFLT-1 was increased by ˜15-fold within 15 minutes of occlusion (20.3±10 versus 238.5±31 pg/mL, respectively, p<0.001) and then remained significantly elevated at 30 and 60 minutes after septal artery occlusion (FIG. 12). Within 24 hours of arterial occlusion, sFLT-1 levels were within normal limits. In the same samples, myoglobin and CK-Mb levels did not reach levels above 3 times the pre-ablation value until 30 minutes after septal artery occlusion. These findings demonstrate that sFLT-1 exhibits very rapid release kinetics, which exceeds the ability of biomarkers of myocardial necrosis for early detection of coronary artery occlusion.

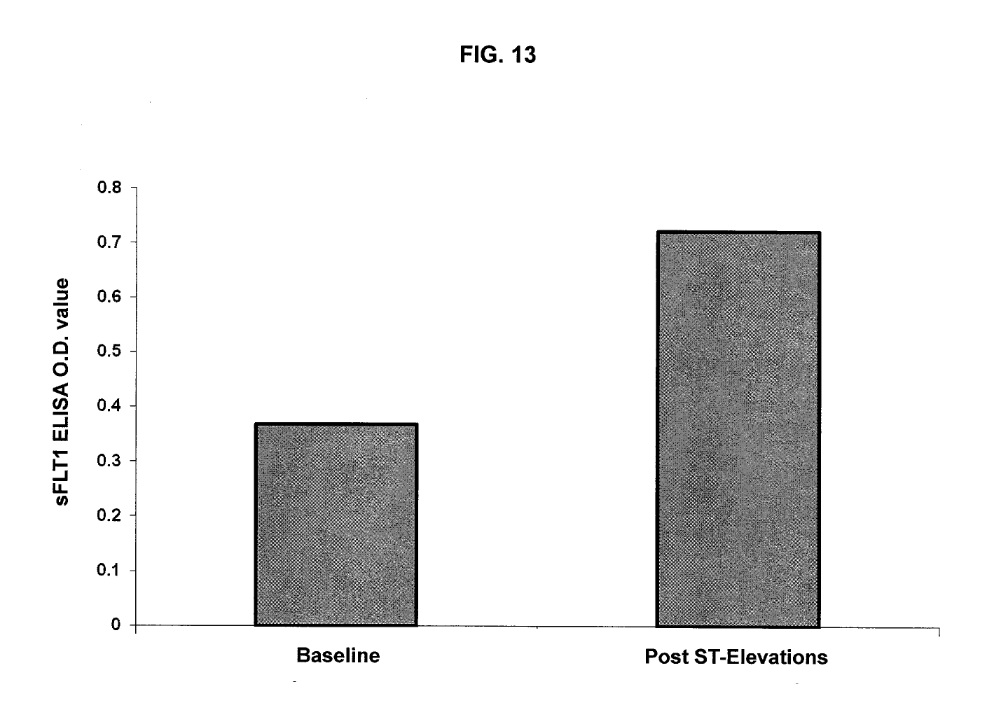

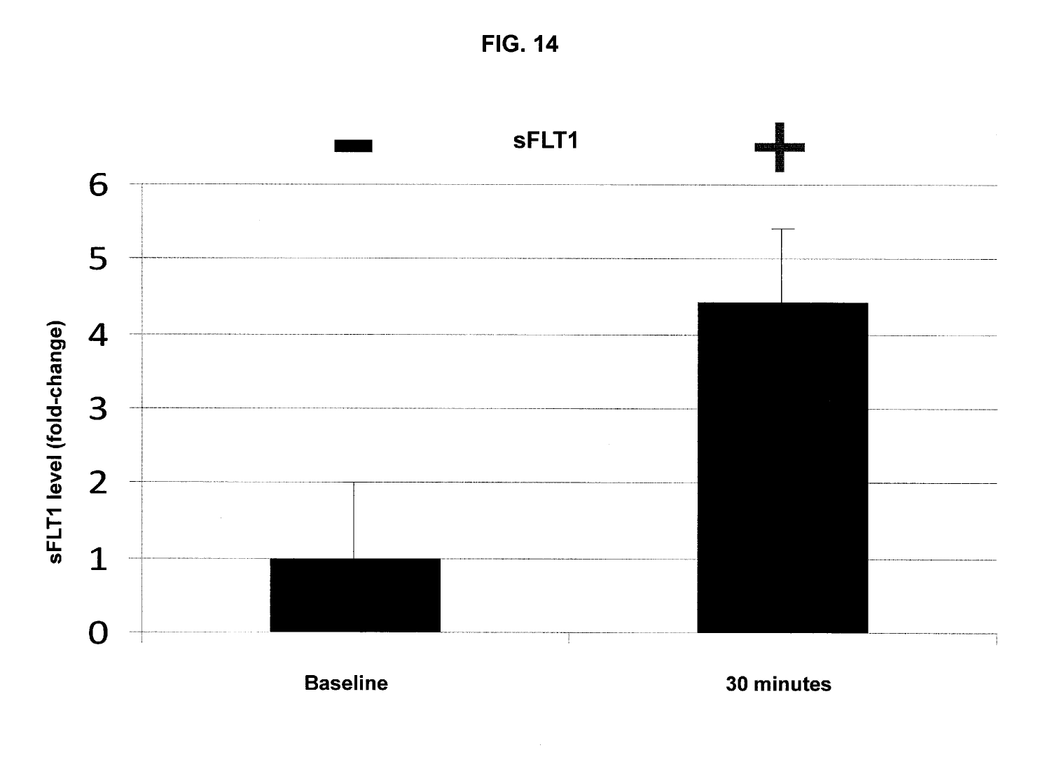

In another example, sFLT-1 levels were determined in a patient that developed ST-elevations during a coronary stenting procedure. As shown in FIG. 13, sFLT-1 levels doubled within minutes of arterial occlusion. FIG. 14 shows that sFLT-1 levels remained elevated 30 minutes after occlusion.

These findings have several clinical implications. In one example, the ability to diagnose STEMI within minutes of symptom onset may improve clinical outcomes as therapeutic interventions are employed without delay. In another example, because sFLT-1 originates from the endothelium, myocyte necrosis is not required for detection of sFLT-1 in the serum, indicating that sFLT-1 may serve as a biomarker of endothelial hypoxia without myocyte necrosis. Furthermore, the detection of elevated levels of sFLT-1 allows for discrimination between a diagnosis of acute vascular occlusion versus another form of myocyte injury (e.g., myocarditis). In a final example, the ability to detect reocclusion of a coronary vessel after successful reperfusion is presently limited by the delayed kinetics of current biomarkers. Since sFLT-1 levels normalize within 24 hours, measurement of sFLT-1 levels enables diagnosis of acute coronary reocclusion.

### Other Embodiments

From the foregoing description, it is apparent that variations and modifications may be made to the invention described herein to adopt it to various usages and conditions. Such embodiments are also within the scope of the following claims.

All publications, patent applications, and patents mentioned in this specification are hereby incorporated by reference to the same extent as if each independent publication or patent application was specifically and individually indicated to be incorporated by reference.

What is claimed is:

1. A method of diagnosing reocclusion of a blood vessel after reperfusion of said blood vessel in a subject, said method comprising the steps of:
(a) obtaining a biological sample from said subject, wherein said biological sample is obtained after reperfusion of a blood vessel; and
(b) measuring the level of sFLT-1 present in said biological sample,
wherein an increase in the level of sFLT-1 in said subject compared to the level of sFLT-1 in a subject not suffering from reocclusion of a blood vessel indicates reocclusion of a blood vessel in said subject.

2. The method of claim 1, said method further comprising:
(c) obtaining a second biological sample from said subject, wherein said second biological sample is obtained after said biological sample of step (a); and
(d) measuring the level of sFLT-1 present in said second biological sample,
wherein an increase in the level of sFLT-1 in said second biological sample compared to said biological sample of step (a) indicates reocclusion of a blood vessel in said subject.

3. The method of claim 2, wherein said second biological sample is obtained 3, 6, 12, 24, 48, or 72 hours after obtaining said biological sample of step (a).

4. The method of claim 1, wherein said blood vessel is a coronary blood vessel.

5. The method of claim 4, wherein said coronary blood vessel is an artery.

6. The method of claim 1, wherein said reocclusion occurs in a subject with a cardiovascular condition.

7. The method of claim 6, wherein said cardiovascular condition is acute coronary syndrome, atherosclerosis, transient ischemic attack, systolic dysfunction, diastolic dysfunction, aneurysm, aortic dissection, myocardial ischemia, angina pectoris, stable angina, unstable angina, acute myocardial infarction, acute ST-segment elevation myocardial infarction (STEMI), acute non-STEMI, congestive heart failure, dilated congestive cardiomyopathy, hypertrophic cardiomyopathy, restrictive cardiomyopathy, cor pulmonale, arrhythmia, valvular heart disease, endocarditis, pulmonary embolism, venous thrombosis, and peripheral vascular disease.

8. The method of claim 7, wherein said cardiovascular condition is acute STEMI.

9. The method of claim 1, further comprising measuring the level of one or more additional biomarkers in said biological sample.

10. The method of claim 9, wherein said one or more additional biomarkers are selected from the group consisting of annexin V, β-enolase, cardiac troponin I, cardiac troponin T, creatine kinase-Mb, glycogen phosphorylase-BB, heart-type fatty acid binding protein, C-reactive protein, growth differentiation factor 15, phosphoglyceric acid mutase-MB, S-100ao, myoglobin, actin, myosin, and lactate dehydrogenase.

11. The method of claim 1, wherein said biological sample is obtained after surgery.

12. The method of claim 1, wherein said biological sample is blood, serum, or plasma.

13. The method of claim 1, wherein said level of sFLT-1 in said subject not suffering from reocclusion of said blood vessel is between 5-20 pg/ml.

14. The method of claim 1, wherein said level of sFLT-1 in said subject diagnosed with said reocclusion of said blood vessel is between 100-400 pg/ml.
